(19) United States
(12) Patent Application Publication
(10) Pub. No.: US 2008/0259321 A1
(43) Pub. Date: Oct. 23, 2008

(54) System and Method for Rapid Reading of Macro and Micro Matrices

(75) Inventors: Peter Lea, Toronto (CA); Nicole Szabados Haynes, Richmond Hill (CA); Colin Dykstra, Toronto (CA); Shi-Fa Ding, Toronto (CA); Uwe D. Schaible, Ancaster (CA); Norman H. Von Styp-Rekowski, Oakville (CA)

Correspondence Address: TRASK BRITT, P.O. BOX 2550, SALT LAKE CITY, UT, 84110, US

(73) Assignee: Umedik Inc., Toronto, ON (CA)

(21) Appl. No.: 11/632,983

(22) PCT Filed: Jul. 19, 2005
(86) PCT No.: PCT/CA05/01134

§ 371 Date: Mar. 3, 2008

(30) Foreign Application Priority Data
Jul. 20, 2004 (CA) 2475191

(51) Int. Cl. G01J 1/00; G01N 33/48; G01J 1/58

(52) U.S. Cl. 356/213; 702/19; 250/458.1

(57) ABSTRACT

An analyte reading system which includes a reader unit for rapidly detecting and evaluating the outcome of an assay to measure the presence of analytes in a sample. Quantitative and qualitative measurements of analyte concentration in a sample may be rapidly obtained using the reader device with algorithms which ascertain the nature of the assay and perform a comparison against a calibration sample. The reader device scans preset areas of an assay device in order to provide focal points for the reader device and evaluate the volume of the test sample in the assay device. The reading portion of the assay slide has at least one test dot for detecting the presence of the analyte and the signal intensity of the labelled analyte, and processes the detected signal using an algorithm which provides an accurate output measurement indicating the quantity of the analyte in the test sample. The reader device can read the analyte as a random array format, print and read the analyte to be measured in a fixed array format, and print and read the analyte in a hybrid format consisting of both fixed and random arrays.

### FIELD OF THE INVENTION

The present invention relates to a device and the reading and data analysis of an assay device for identification and quantification of analytes.

### BACKGROUND OF THE INVENTION

Micro matrices of bacteria and macro matrices of their respective toxic proteinaceous contaminants account for several million cases of food-related illness and about 9,000 deaths per year in the United States. Contaminated processed food, poultry and meat products etc. are a major cause of these deaths and illnesses. The five most common pathogens infecting food products and especially poultry and meat products are E. coli O157:H7, Salmonella species, Listeria species, Listeria monocytogenes and Campylobacter jejuni.

Similarly, contamination of water supplies also causes illness and death. The United States Environmental Protection Agency has determined that the level of E. coli in a water supply is a good indicator of health risk. Other common indicators are total coliforms, fecal coliforms, fecal streptococci and enterococci. Currently, water samples are analyzed for these micro-organisms using membrane filtration or multiple-tube fermentation techniques. Both types of tests are costly and time consuming and require significant handling. They are not, therefore, suitable for field-testing.

Accordingly, to prevent infection of consumers through contaminated food and water and detection of many disease conditions there is a need for the accurate and rapid identification of micro-organisms and markers of the health of a patient. The accurate, rapid detection and measurement of micro-organisms, such as bacteria, viruses, fungi or other infectious organisms and indicators aggregates in food and water, on surfaces where food is prepared, and on other surfaces which should meet sanitary standards is, therefore, a pressing need in industrial, food, biological, medical, veterinary and environmental samples. Further, in routine inspection of industrial products for microbiological contamination there is a need for the early detection of contamination to permit rapid release of safe products, and for the rapid, accurate detection and measurement of micro-organisms which are not pathogenic but have a role in the determination of a product's shelf life.

A variety of assay methodologies have been used for determining the presence of analytes in a test sample. Assays for detecting micro-organisms generally require that the samples be grown in culture. In this assay, the typical practice is to prepare a culture growth medium (an enrichment culture) that will favour the growth of the organism of interest. A sample such as food, water or a bodily fluid that may contain the organism of interest is introduced into the enrichment culture medium. Typically, the enrichment culture medium is an agar plate where the agar medium is enriched with certain nutrients. Appropriate conditions of temperature, pH and aeration are provided and the medium is then incubated. The culture medium is examined visually after a period of incubation to determine whether there has been any microbial growth. It could take several days to obtain results and requires a technician to read the agar plates by visual inspection. Attempts to identify the organisms of interest can lead to additional error and delay in time to test results.

Many disease conditions, such as bacterial and viral infections, many cancers, heart attacks and strokes, for example, may be detected through the testing of blood and other body fluids, such as saliva, urine, semen and feces for markers that are known to be indicative of specific conditions. Early and rapid diagnosis may be the key to successful treatment. Standard medical tests for quantifying markers, such as ELISA-type assays, are time consuming and require relatively large volumes of test fluid.

There are presently many examples of one-step assays and assay devices for detecting analytes in fluids. One common type of assay is the chromatographic assay, wherein a fluid sample is exposed to a chromatographic strip containing reagents. A reaction between a particular analyte and the reagent causes a colour change on the strip, indicating the presence of the analyte. In a pregnancy test device, for example, a urine sample is brought into contact with a test pad comprising a bibulous chromatographic strip containing reagents capable of reacting with and/or binding to human chorionic gonadotropin (“HCG”). The urine sample moves by capillary flow along the bibulous chromatographic strip. The reaction typically generates a colour change, which indicates that HCG is present. While the presence of a quantity of an analyte above a threshold level may be determined, the actual concentration of the analyte is unknown. Accordingly, there is a risk that a pathogen may be present below a level sufficient for either the test to detect its presence, or for the individual assessing the test strip to visually observe the confirming colour change of the test strip.

Assays have been developed for providing a quantitative measure for the presence of pathogens or analytes of interest. In such a typical test assay, a fluid sample is mixed with a reagent, such as an antibody, specific for a particular analyte (the substance being tested for), such as an antigen. The reaction of the analyte with the reagent may result in a colour change that may be visually observed, or release of chemiluminescent, bioluminescent or fluorescent species that may be observed with a microscope or detected by a photodetecting device, such as a spectrophotometer or photomultiplier tube. The reagent may also be a fluorescent or other such detectable-labelled reagent that binds to the analyte. Radiation that is scattered, reflected, transmitted or absorbed by the fluid sample may also be indicative of the identity and type of analyte in the fluid sample.

In a commonly used assay technique, two types of antibodies are used, both specific to the analyte. One type of antibody is immobilized on a solid support. The other type of antibody is labeled by conjugation with a detectable marker and mixed with the sample. A complex between the first antibody, the substance being tested for and the second antibody is formed, immobilizing the marker. The marker may be an enzyme, or a fluorescent or radioactive marker, which may then be detected.

A large variety of assays and other specific binding assay is already known. These assays essentially are qualitative lateral flow devices to be read by eye and quantitative assays which are to be read by generic reading devices.

Examples of such assays and the materials used are described in detail in reference texts “Principles and Practice of Immunoassay”, (Price C. P. and Newman D J, Eds.) Stockton Press 1997, ISBN 1-56159-145-0; “The Immunoassay Handbook”, (Wild, D. Ed.) Nature Publishing Group 2001, ISBN 0-333-72306-6 and “Protein Microarrays”, (Schena, M. Ed.) Jones and Bartlett Publishers 2005, ISBN 0-7637-3127-7.

To date, emphasis has predominantly been placed on the development of respective assays, when co-development between assay device and an optimal reading of the assay in a reading device is needed. The required reader device is not only a simple imaging relay device, but should have the capability to interface and interactively, recognize the dependent assay device. In order to quantitatively measure the concentration of an analyte in a sample and to compare test results, it is usually necessary to either use a consistent test volume of the fluid sample each time the assay is performed or to adjust the analyte measurement for the varying volumes. Incorporation of specific algorithms, micro-fluidics and ergonomics should provide an integrated system for application of a method when reading micro and macro matrices.

There is need of a system and method which can efficiently, rapidly and accurately read an assay for determining the presence of analytes in a sample and for determining the quantity of respective analytes in the sample in an efficient, simple and reliable manner.

### SUMMARY OF THE INVENTION

The present invention provides an analyte reading system which includes an analyte reader device for rapidly detecting and measuring the presence of analytes of test sample in a co-dependent assay device. Quantitative and qualitative measurements of analyte concentration in a sample may be rapidly obtained using the reader device with preset algorithms which also ascertain the nature of the assay being read, provide controls and can prevent erroneous duplication of measurement of that assay.

According to a method of the present invention, the reader device can detect from a reading area of an assay device, control reference spots from which the system can calculate or ascertain the nature of the assay or assays conducted in the assay device, meter the volume of test sample and read simultaneous reference calibration curves in the assay device. The calibration matrices, which are measured within the assay device as the test sample concentrations are measured, allows the reading device to generate respective calibration curves to be used in the deriving the actual concentrations of the unknown analytes contained in the test sample.

According to another aspect of the present invention, the reader device can scan preset areas of an assay device in order to provide focal points for the reader device and evaluate the volume of the test sample in the assay device. This aspect of the invention permits the reader device to adjust the analyte measurement for varying volumes.

According to another aspect of the present invention, there is provided a reading system for reading and measuring the outcome of an assay in an assay device containing a labelled analyte, comprising a positioning stage for holding the assay device in a desired position, a light sensor, an optical system comprising an excitation light source for illuminating a labelled analyte, and a dichroic mirror for reflecting excitation light to the analyte and light emitted by the dye to pass through to the light sensor, and a computer for processing the signal detected by the light sensor to generate a measurement of analyte density on a detected portion of the assay device.

According to yet another aspect of the present invention, there is provided a method of reading an assay device containing a labelled analyte, comprising the steps of illuminating a portion of the assay device containing a test sample, detecting an intensity of light emitted by the test sample in a single image field, and generating a measurement of analyte density in the test sample based on said intensity detection.

According to another aspect of the present invention, there is provided, a method of reading an assay device containing a fluorescently labelled analyte, comprising the steps of illuminating a portion of the assay device containing a test sample of unknown analyte density, illuminating a portion of the assay device containing a calibration sample of known analyte density with an excitation light, detecting an intensity of light emitted by the unknown concentration of test sample and an intensity of light emitted by the known concentration of calibration sample in a single image field, and comparing the intensity of light emitted by the unknown concentration of test sample to the intensity of light emitted by the known concentration of calibration sample to generate a measurement of analyte density in the test sample.

The present invention thus provides an analyte reading system consisting of a unit for reading and measuring the qualitative and quantitative outcome of an assay in an assay device for a labelled analyte, comprising an X-Y-Z positioning stage for holding the assay device in a desired location, a light sensor, and an optical system comprising an excitation light source for illuminating a labelled analyte, and a dichroic mirror for reflecting excitation light to the analyte and emittor radiation to pass through to the light sensor.

The present invention further provides an analyte reading system for measuring the outcome of an assay in an assay device containing a fluorescently labelled analyte, comprising a positioning stage for holding the assay device in a desired position, a light sensor, an optical system comprising an excitation light source for illuminating a fluorescently labelled analyte, and a dichroic mirror for reflecting excitation light to the analyte and light emitted by the fluorescent dye to pass through to the light sensor, and a computer for processing the signal detected by the light sensor to generate a measurement of analyte concentration on a detected portion of the assay slide.

The present invention further provides a method of reading an assay device containing a fluorescently labelled analyte, comprising the steps of a. illuminating a portion of the assay slide containing a test sample; b. detecting an intensity of light emitted by the test sample in a single image field; and c. generating a measurement of analyte density in the test sample based on said intensity detection.

The present invention further provides a method of reading an assay device containing a fluorescently labelled analyte, comprising the steps of: a. illuminating a portion of the assay slide containing a test sample of unknown analyte density and a portion of the assay slide containing a calibration sample of known analyte density with an excitation light; b. detecting an intensity of light emitted by the test sample and an intensity of light emitted by the calibration sample in a single image field; and c. comparing the intensity of light emitted by the test sample to the intensity of light emitted by the calibration sample to generate a measurement of analyte density in the test sample.

### BRIEF DESCRIPTION OF THE DRAWINGS

In drawings which illustrate by way of example only a preferred embodiment of the invention,

FIG. 1 is a schematic view of a Reader Device of the present invention;

FIG. 2 is a flow-chart of the image processing in the Reader Device of the present invention;

FIG. 3 is an a micrograph of a focus spot in the Assay Device as read by the reader device of FIG. 1;

FIG. 4 shows a map of the virtual window assignment for the reading area of the Assay Device shown in FIG. 7;

FIG. 5 illustrates the Assay Device identification arrays and encoding algorithm;

FIG. 6 illustrates the Assay Device control array;

FIG. 7 is a schematic drawing of a reader-compatible Assay Device;

FIG. 8 illustrates a calibration array and a capture array in the viewing area of an Assay Device;

FIG. 9 is an example of typical calibration and capture arrays;

FIG. 9A is a graphical representation of a Fixed Array layout;

FIG. 10 plots the data for Fluorescence Response against concentration for Calibration and Capture Array responses;

FIGS. 11A and 11B show the calibration arrays compared to patient plasma testing for exposure to Toxoplasma gondie;

FIGS. 12A and 12B are schematic illustrations of an Acquired Pathogen Array (APT) and a Protein Array, respectively;

FIG. 13 illustrates core sections of Tumour Tissue section arrays; and

FIG. 14 is a schematic view of an analyte reader system of the invention incorporating the Reader Device of FIG. 1.

### DETAILED DESCRIPTION OF THE INVENTION

The present invention provides an analyte reading system and method for the rapid reading of macro and micro matrices. A macro matrix consists of objects to be detected and measured when the objects are molecular aggregates ranging in size from about 5 μm (micrometers) to about 1000 μm. These objects are usually planar, essentially two dimensional or flat spots that are attached to a substrate contained in the assay device. A macro matrix is defined as a “fixed macro array” containing multiple spots, each located at known X-Y locations in the assay device. The locations of individual spots that make up an array, have pre-determined centre-to-centre spacing. Location of the spots which make up the arrays in a matrix is found automatically by the reading device from a primary reference spot also on the assay device. The reader focuses on the spots in the plane of attachment. A “fixed macro array” is further characterized into being a “fixed macro test array” for detection and measurement of unknown concentrations of test sample and “fixed macro calibration array” for the generation of respective calibration curves from known concentrations of calibrators. Both types of arrays are read by the reader within the same assay device for each test to obtain accurate quantitative measurement of analyte represented in the molecular aggregates.

A micro matrix ranges in size from about 0.25 μm to about 5 μm. These objects are usually discrete micro-organisms or particles that tend to be randomly distributed in three dimensional space defined by the volume of test fluid in the assay device. A micro matrix is defined as a “random micro array” containing free floating, three-dimensional objects suspended in three-dimensional space.

### EXAMPLE 1

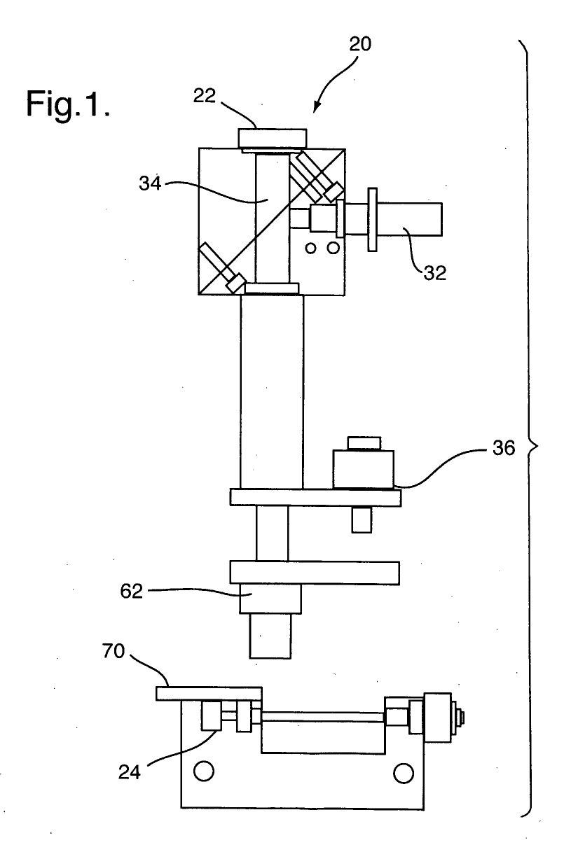

As illustrated in FIG. 1, the preferred embodiment of the analyte reading device 20, has a fully automatic analytical interface with a co-functional assay device and an imaging device such as a CCD camera 22 which transmits signals to a general purpose computer integrated into the system. The reader device 20 has a stage 24, stage movement (X and Y axes) for assay device positioning 70 and auto-focusing (Z axis) for image clarity and resolution 36, controlled by servo motors through a suitable user interface, such as a touch-pad or touch-screen control board.

In the preferred embodiment the computer is programmed to process the signal returned by the CCD camera 22 to provide accurate assay identification and results, as described in detail below; however the computer may also be programmed to control the functions of the analyte reading unit via user displays and touch-screen activation of functions. The reader device has an optics assembly 62. Optics assemblies known in the art may be used for the purposes of the present invention. The microscope 20 also has a dichroic mirror 34 and an auto-focus mechanism 36. A laser 32 is connected to the dichroic mirror 34. The options assembly 30, shown in FIG. 14, controls the laser 32 that is adapted to apply energy to the dichroic mirror 34 that forms part of the microscope 20.

### EXAMPLE 2

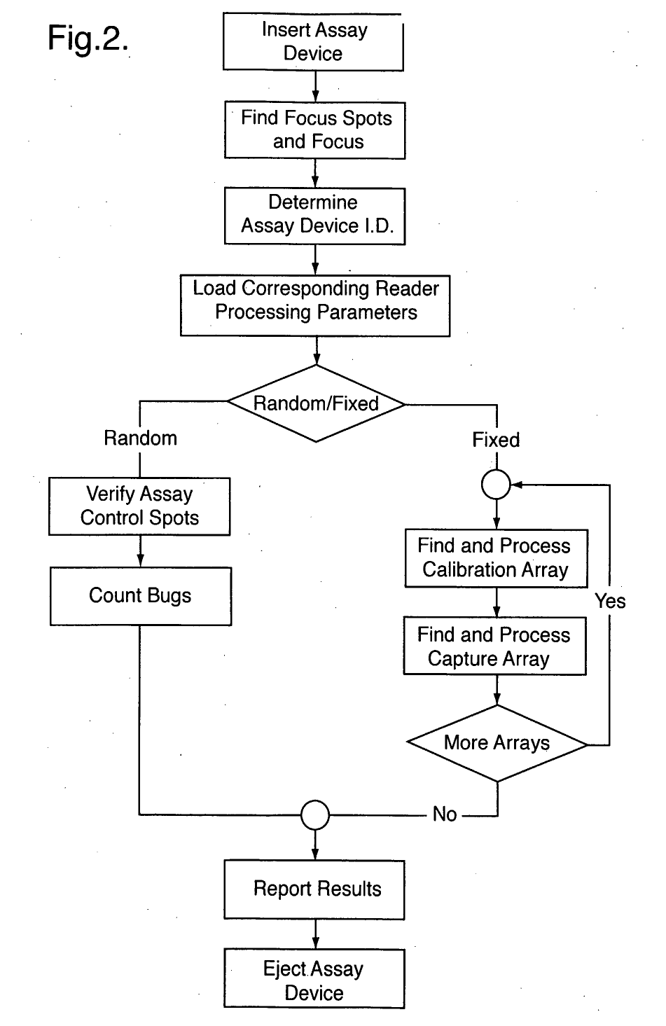

The flowchart illustrated in FIG. 2 outlines the processing logic of the reader device and the Assay Device when the test sample has been prepared using the Assay Device assembly. Once the Assay Device is inserted into the reader and the user presses ‘Begin Scan’ the Reader device X-Y stage draws in the Assay Device to center the viewing area of the assay device.

### EXAMPLE 3

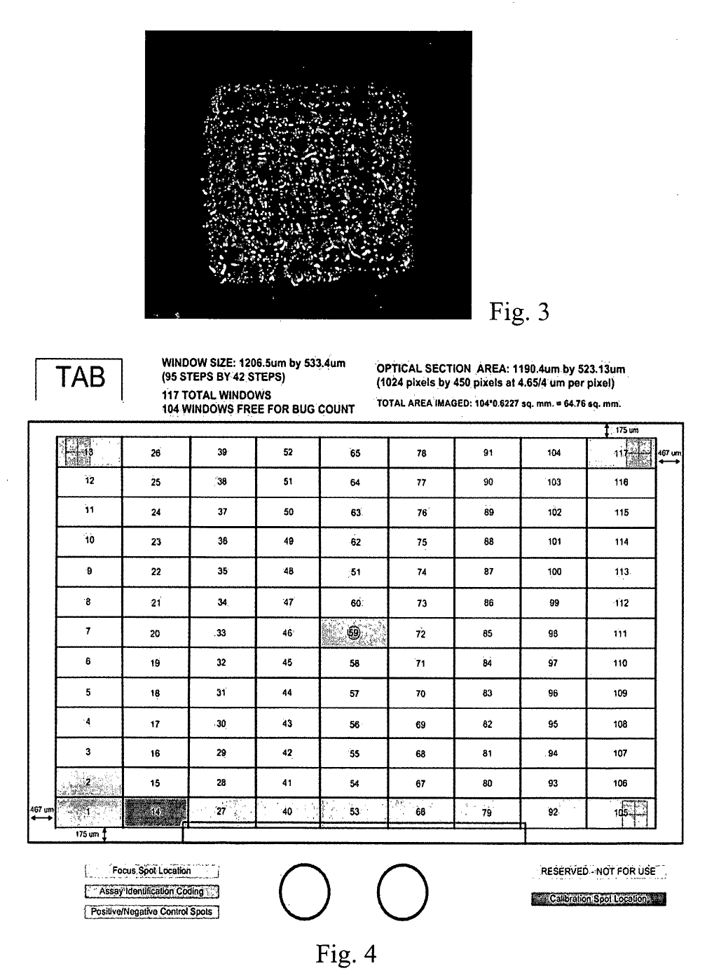

The Assay Device is illuminated under a bright field (LED light source) and a 100×100 pixel image is captured to view and analyze the focus spot, FIG. 3. The Z-axis is adjusted to determine the optimal focus and the Z-axis position is stored as Z1.

The auto-focus spots are molded into the Assay Device at time of manufacturing. These features are approximately 80×80 μm+/−10 um in size also 25-30 μm in focusing depth and are imaged using the full-spectrum LED light source.

The stage auto-ranges and moves so that the image center is located at the exact center of the focus spot in window 117. Repeating the 100×100 pixel image capture and analysis with Z-axis adjustment again focuses the image. The new Z-axis position is stored as Z2. Finally, the stage is moved so that the image center is located at the exact center of the window 105. The image is focused again and the new Z-axis position is stored as Z3.

An optimal focus plane is then calculated using Z1, Z2, and Z3, after which the Z-axis is calculated for the optimal focus value for each focus spot location.

The stage is then moved to the center of window 1 which contains the Assay Device Identification array and a full 1024×768 pixel window is captured under a laser illumination. The captured image is analyzed and the Assay Device Assay Type is determined. The assay type identifies the analyte organism and whether the assay is a fixed or random array. The stage is then moved to window 14 where a duplicate assay identification array is located. The second array is imaged and analyzed and the results are compared to ensure that the correct assay type has been determined. Should the two differ, the test will halt and the operator will be notified.

Based on the assay type, the Reader will then either process the Assay Device as a random array (typically microbial identification and quantification) or will begin fixed array processing.

In operating the system, a user places an assay device that is to be read onto the stage 24, FIG. 1. The system then applies an initialization and an auto-calibration routine. The auto-calibration is referenced to an emission standard, which, under software control, tests and calibrates the optics assembly as needed. The performance levels of the instrument are monitored via remote access, e.g. the internet, and may be adjusted also by remote control.

### EXAMPLE 4

### Assay Device Virtual Window Construct

The reader is interactive with the assay device in that the viewing area of the assay device is partitioned into virtual areas of viewing or imaging.

FIG. 4 shows the layout and numbering of the virtual windows ascribed to the viewing area of the assay device. The locations of the special purpose windows are highlighted in FIG. 4.

### EXAMPLE 5

### Assay Device Identification Array and Encoding Algorithm (Virtual Windows 1 and 14)

The Assay Device Identification Array is a 4×3 grid of 80 μm+/−10 μm diameter spots arrayed on a 150 μm+/−10 μm pitch. The grid is left-justified and placed in virtual window 1 with a duplicate array replicated in virtual window 14.

The Assay Device Identification Array is comprised of two elements—a reference column of three spots that will always be present and a 3×3 array that is a binary encoding that, when decoded, will give an Assay Device ID that uniquely identifies each type of assay.

The binary encoding will be from least significant to most significant from left to right across the three columns. To increase the reliability of the identification algorithms, the binary values “000” and “111” will not be permitted in any column. Therefore there are 6×6×6=216 valid Assay Device IDs. Should additional values be required in the future, there is space to add additional columns to the array. Adding another column of 3 spots will produce 1296 valid Assay Device IDs. This is effectively using Base 6 to encode the values, with an offset of 1 (i.e. “0” will never be valid).

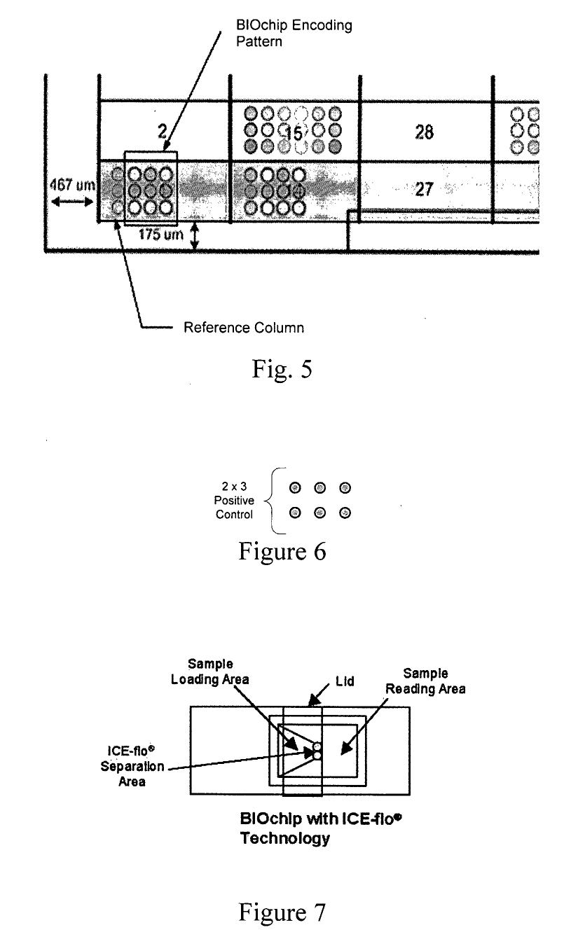

FIG. 5 encodes 010 110 010 which translates to Assay Device ID #262.

The purpose of the reference column of three spots is to ensure that the Assay Device ID software always locates the left edge of the array. Assay Device ID values are not allowed to be “111” to ensure that the algorithm can differentiate a valid numeric column from the reference column. Similarly, “000” is not permitted so that the algorithm will always have at least one spot in a column. The unique identification code can be obtained from the specific Product Plot allocated in the product part number. e.g. Listeria Genus has Plot Number LIG02001 with ID of 111.

### EXAMPLE 6

### Assay Control Array (Virtual Window 59)

The Assay Control Array, located in Window 59, is present only in Random Array assays. It consists of a left justified 2×3 array of 85 μm+/−10 μm diameter spots on a 150 μm+/−10 μm pitch.

The Assay Control Array is used as a positive control to ensure that the assay is functioning correctly. Each control spot is composed of denatured organisms of the assay's analyte. For example, the control spot on a Listeria Assay are composed of denatured Listeria.

When the sample is introduced into the Assay Device assembly, the excess labeled antibodies will react with and collect on the control spots. The window is imaged using the laser excitation source and the assay is presumed to have worked correctly if the spots are emitting signal. The control spots will not emit any signal if an incorrect sample preparation is used.

The combination of imposing sequential, dedicated areas of illumination to be examined, allows only the window under examination to be illuminated. The surprising benefit is that while this window is being examined, the remaining viewing area is not being irradiated and therefore preserves optimal detection output. This results in specimen preservation which is in direct contrast to standard readers which expose the whole viewing area to continually scanning irradiation. The assay device preferably has at least one identification coding dot that is detected by the reader system to provide identification of which assay is being tested and ensure that the appropriate sub-routine or multiple sub-routines for image analysis is read and accordingly which routines and calculations need to be carried out.

In one embodiment of the invention the analyte reading system is designed to detect micro-organism antigens marked or coated with an indicator such as a fluorescent labelled antibody. In this embodiment the analyte reading system can be used to determine the concentration in a given sample of the micro-organism antigen. The antigen concentration, which can be used as a measure of the micro-organism concentration from a sample, such as a food sample, can then be compared with an acceptable analyte concentration limit and a pass/fail response reported to the user.

In this embodiment of the invention the analyte reader unit is adapted to read and detect specifically labelled analytes in an assay slide or assay chip into which the analyte sample is placed. One fluorescent dye suitable for labelling bacteria for use in the designed assay chip is Alexafluor™ 647 nm dye. It is the assay chips which are presented to the analyte reader for scanning. One skilled in the art will appreciate that alternatives to fluorescent labelling can also be used. Whichever labelling system is used, the light source (which may include electromagnetic radiation ranging from ultraviolet to infrared) for imaging and the detector must be matched, and may be collectively referred to as the imaging system.

### EXAMPLE 7

### Operation of the Random Array Assay Device Format

The Random Array reading format is technology unique to the present invention. Pathogens are tagged with fluorescent dye markers, including use of organism-specific antibodies, receptor binding and other methods known in the art. The now fluorescing pathogens are directly enumerated in a known sample volume, resulting in accurate, quantified test results. The random array format uses also ELISA immuno-chemistry for “on-chip” calibration purposes and as positive control. The system actually counts individual micro-organisms to establish the concentration of micro-organisms in the tested sample. The accuracy of this count when compared to the current agar plating and incubation leading to a physical count of colonies grown gold standard method, has confirmed a 1:1 concordance.

Both the Random and Fixed array share a common system platform—the Assay Device, Analyte labeling and the Reader Device. The assay device, for use with reader, in the preferred embodiment has the following main characteristics:
All required chemical compounds needed to process a sample are contained in a single-use, disposable, Analyte labeling applicator. No specialized training is required to use the assay device.
The liquid sample is drawn from the sample loading area into the sample reading area by means of almost instantaneous fluid transfer.
The fluid sample is optionally processed through a tunable dynamic separation matrix during the fluid transfer phase to exclude background contamination.
The amount of test volume contained in the sample reading area is self-metering and has a fixed volume. Once the sample reading area is filled, no additional fluid is drawn from the sample loading area.
All Assays are automatically self-calibrating.
All Assays are single use. Once a chip has been read and the data processed, it is automatically marked in a way that will prevent the reader from processing a chip a second time.

In the embodiment for reading and counting the actual number of specific microbes contained in a known sample volume of fluid as measured by the assay device, the optical imaging system sequentially examines cylindrical fluid volumes of sample held under the viewing area of the Assay Device. The reader proceeds to scan and count the micro-organisms contained in each of these “optical volumes” and calculates and displays a requisite concentration upon completion of the window scans. The virtual windows, or 3-D matrix volumes, are created by the x/y co-ordinates which drive the Reader stage. The interaction of the reader and the assay device therefore creates virtual windows as an x/y matrix, in which each virtual window, or optical volume, contains signal generating micro-organisms in the format of a three-dimensional random array within the optical volume. Because these micro-organisms or particles are not fixed to a substrate but are in suspension and because they are generally less than about 5 micrometers in size, they are defined as “random micro matrices”. An added advantage of rapid, automatic, sequential optical volume imaging, is that particle counting error and background is significantly reduced as a sample optical volume is being scanned because the reader detects and measures micro-fluidic parameters to also discriminate true micro-organisms from random background contamination. These parameters include signal to noise ratio analysis, fitting the detected micro-organisms into size categories, background subtraction and particle movement analysis.

### EXAMPLE 8

### Reader Processing of Random Array Assay Format

The Random Array method is used test for the presence of pathogenic organisms. The organism is tagged with a fluorescent dye and the number of organisms present in a sample is directly enumerated by the Reader.

Imprinted on the underside of the sample viewing area are six positive-control dots. These dots are imprinted at time of manufacture with the pathogen of interest. During the fluid transfer phase, significant populations of the loose pathogen-specific antibodies are bound to the positive-control dots. This serves as the positive-control aspect of the test.

The Assay Device is then inserted into the reader for automated analysis.

Printed on the Assay Device is an assay-specific identifier. The reader seeks to the specific location of the Assay Device containing the assay-specific identifier and loads any pathogen-specific analysis routines. The reader then locates and confirms that the positive-control dots have been tagged with the loose antibodies. If an incorrect analyte labeler has been used to dispense the sample, the reader will recognize that the test has been compromised and the test run will terminate with an appropriate notification message.

Once the positive-control test has completed, the reader proceeds to processing the chip and enumerating the pathogenic organisms tagged with fluorescent-dye (via the pathogen-specific antibodies). The processing steps conducted are as follows:
The sample viewing area is divided into more than 100 individual virtual sample windows. These sample windows are referred to as optical volumes.
The reader detects and enumerates the number of dye-tagged pathogens found in each optical volume.
Given that each optical section is of a known volume, it is, therefore, possible to calculate and quantify the number of pathogens found in the sample.
The reader processes the 100+ optical sections in approximately 4-5 minutes and reports the number of Pathogens per milliliter to the operator on the front panel.
Given that the reader is able to average the detected pathogen population over a significant number of optical volumes, a high degree of confidence level is achieved.

The results are available for reporting to QA systems or for hard copy printout.

In a preferred embodiment the optical system consists of five parts: a light source such as a laser light source, a light emitting diode (LED) ring light source, a filter cube, a microscope objective lens, and an optical tube with focussing. In this embodiment the laser light source preferably has a peak spectral emission at 635 nm. The laser spectral emission at 635 nm then passes through an excitation filter of the filter cube. This excitation filter is used to control the bandwidth and wavelength of light that will reach the assay chip assay chip in the analyte reader unit. In this embodiment the excitation filter allows only the 635 nm emission line from the laser light source to be passed to the filter cube's dichroic mirror, which then reflects this light down the axis of the optical tube towards the microscope objective lens. The laser light is focused on the assay chip assay chip by the microscope objective lens and causes the labelling marker, in this embodiment the Alexafluor™ 647 nm fluorescent dye attached to the antibody bound (directly or indirectly) to the analyte to fluoresce and emit light with a peak intensity at 668 nm.

In a preferred embodiment of the invention, the assay chip containing the labelled test sample also has focus spots. To ensure accuracy in this embodiment of the invention, the analyte detector device ideally will auto-focus the optical system by reference to the focus spots carried on the assay chip. When the analyte detector device is focussing by imaging the focus spots on the assay chip in this embodiment the laser light source used to provide the excitation of the labelled sample is prevented from illuminating the assay chip. This may be achieved in a variety of ways such as switching off the laser or blocking the light from the laser light source from entering the filter cube. The bright field illumination of the assay chip for imaging of the focus spots in this embodiment is provided by side illumination of the assay chip from the LED ring light source. In one embodiment the bright field side illumination of the assay chip is provided by four Lumex™ SSL-LX5093SRC/E 3500mcd 660 nm high brightness LEDs which are used in an LED ring around the microscope objective.

A suitable microscope objective lens for this embodiment of the invention is an Edmund Industrial Optics™ R43-906 4x plan achromatic commercial grade standard microscope objective lens with a working distance of 13.9 mm, which is used to focus an image of the bacteria on the CCD image sensor. This objective lens is designed to produce an image at 150 mm from the top edge of the objective lens.

In this preferred embodiment of the device of the invention, a light-impervious metal optical tube is used to house the optics of the optical reading unit. The purpose of this optical tube is to prevent interference with the detected signal, the excitation light and emitted light by peripheral or external light sources. This optical tube is grooved and the entire assembly is anodized to reduce the reflection of light and prevent reflection of light from the optical assembly directly onto the image sensor. The optical tube provides a conduit for the light from the excitation source and the emitted light from the labelled analyte between the microscope objective lens and the filter cube. In this preferred embodiment the microscope objective lens is attached to the lower end of the optical tube and the filter cube is attached to the upper end of the optical tube. One way in which the filter cube and microscope objective lens can be attached to the optical tube is using threaded attachment.

In the preferred embodiment of the invention a Point Grey Research Dragonfly IEEE-1394 monochrome CCD camera is used to capture images of fluorescing analytes. This camera contains an ICX204AL ⅓″ black and white, 1024×768 pixel, CCD image chip with a pixel size is 4.65 um×4.65 um. The camera in this embodiment is powered from the IEEE-1394 bus and has an interface protocol which is compliant with the IEEE IIDC DCAM V1.3 specification.

Thus, the analyte reading system of the invention can be used to carry out a preferred embodiment of the method of the invention, which comprises illuminating a portion of the assay slide containing a test sample of unknown analyte density and a portion of the assay slide containing a calibration sample of known analyte density with the excitation light; detecting an intensity of light emitted by the test sample and an intensity of light emitted by the calibration sample in a single image field; and comparing the intensity of light emitted by the test sample to the intensity of light emitted by the calibration sample to generate a measurement of analyte density in the test sample.

The optical tube is also provided with a focussing means, in this embodiment using a stepper motor focussing assembly. In an embodiment of the optical tube a Hayden Switch and Instrument™ 26463-12-003 26 mm 12V captive unipolar linear actuator stepper motor is used to move the lower end of the optical tube along the Z-axis. The Z-axis is perpendicular to the plane defined by the assay chip in position on the positioning stage. Thus movement in this Z-axis provides focussing of the microscope objective lens on the assay chip.

A metal frame is used to keep the filter cube, optical tube, image board, and positioning stage in fixed positions relative to each other. The positioning stage is used to move the assay chip in the X-Y plane relative to the microscope objective lens. The Y-axis is along the short dimension of the plane of the assay chip which is perpendicular to the longitudinal axis of the optical tube. The assay chip is inserted onto the positioning stage along the Y-axis of the assay chip. The X-axis is along the long axis of the plane of the assay chip which is perpendicular to the longitudinal axis of the optical tube. The positioning stage can be moved in the X-Y axis using two motors, for example two Hayden Switch & Instrument™ motors. In one embodiment a 26 mm 12V captive unipolar linear actuator stepper motor is used to drive the stage in the X-axis over a 12.7 mm total displacement distance. Similarly, a 26 mm 12V non-captive unipolar linear actuator stepper motor is used to drive the stage in the Y-axis over a 38.1 mm total displacement distance. These examples of motors have a step size of 0.005″ (or approximately 12.7 μm).

The reference (or home) position for the positioning stage is found by moving the positioning stage to a preset position (usually to the limit of its range of movement in the X and Y-axes). At the reference position an electrical contact is established with two detector switches mounted on the positioning stage. One type of detector switch suitable for this application is Panasonic™ Type ESE11HS1. Optionally, the positioning stage can be controllably moved to the locations of several reference marks or points on the assay chip for accurate optical calibration.

### EXAMPLE 9

### Fixed Array Macro Matrices Specifications and Processing

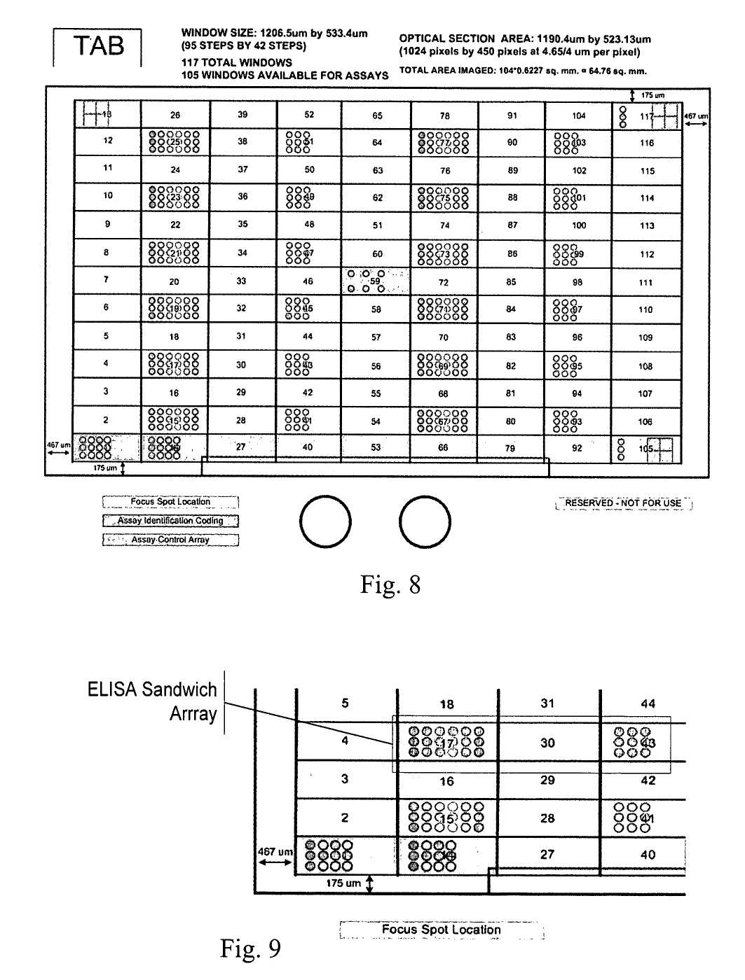

The system of the present invention also reads Fixed Array Macro Matrices and non-biological assays. Each individual fixed array is comprised of two macro matrices—a calibration array which is used as an internal calibrator and a capture array which is used to determine the concentration of the target analyte. Each grid is located in an individual window with an empty window separating them. Therefore, a total of three windows are used for each fixed array. A clear perimeter of windows is reserved on the perimeter of the Assay Device and an empty column and an empty row of windows is reserved between the active windows. This allows a maximum of 12 possible locations for fixed arrays on the Assay Device, as shown in FIG. 8.

### EXAMPLE 10

### Calibration Array

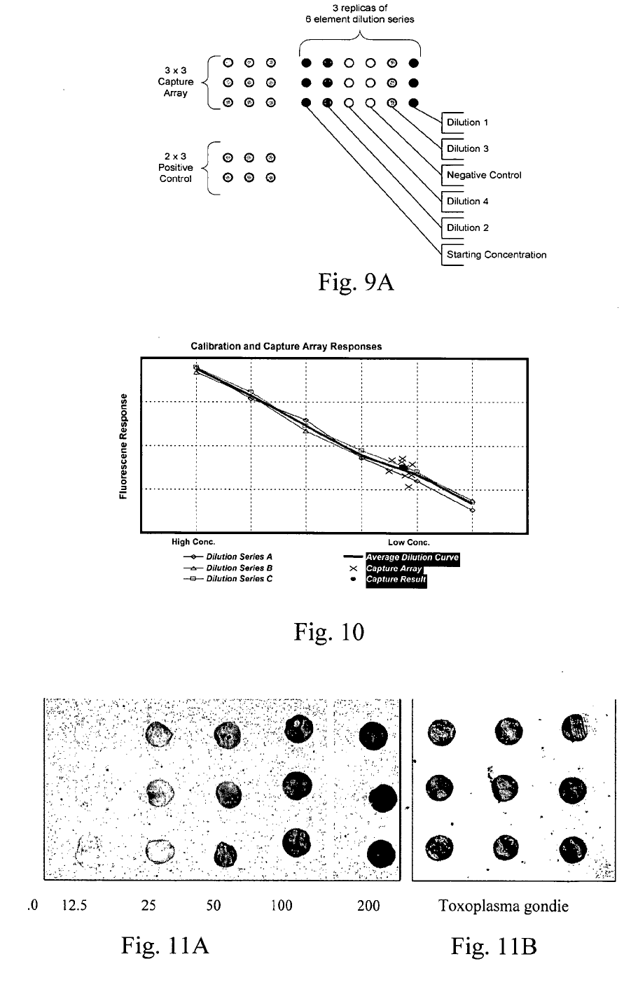

FIG. 9 highlights a calibration array in window 17 and a capture array in window 43.

The calibration array consists of a six-element dilution series of the antigen of interest. The calibration array matrix has three identical replicas of the dilution series. The dilution factor of two is typically used, but factors of 10 can be used. When the analyte is introduced, the excess labeled antibodies bind with the spots in the dilution series spots and fluoresce proportionally when excited by the illumination laser source. The reader takes a single image of the calibration array. The fluorescence intensity for each element of the dilution series from each of the three replicas is measured and a response curve is calculated. This establishes the relationship between the fluorescent intensity of the spots with known antigen concentrations. The calculated response curve captures the antigen of interest and its interactions with the labeled antibody at different concentrations.

Typically, dilution series are arranged in a decreasing or increasing order of concentrations. However, the dilution series in the calibration array is geometrically ordered from the outside inwards. The concentrations, in decreasing order, are allocated to alternating left-most and right-most available columns as described in the following table, typically using 2:1 dilution factor per calibration location:

| Dilution | Concentration | Column |
|---|---|---|
| Original | 100% | 1 (left-most) |
| Dilution 1 | 50% | 6 (right-most) |
| Dilution 2 | 25% | 2 |
| Dilution 3 | 12.5% | 5 |
| Dilution 4 | 6.25% | 3 |
| Negative Control | 0% | 4 |

This arrangement ensures that the most dilute spots are well framed within the higher-dilution spots to facilitate recognition and enhance analysis quality and speed of detection.

FIG. 9A, shows a graphical representation of the Fixed Array layout.

### EXAMPLE 11

### Capture Array

The capture array is a 9-element (3×3) grid of capture antibodies. Each one of these 9 identical replicas is a possible binding site for free floating labeled-antigen analyte complexes. The reader's stage is moved to the capture array and an image is obtained. The fluorescence responses of all 9 replicas are recorded and a representative statistical value (average, mode, or median) is calculated. This value is considered to be the response of the analyte. It is compared to the values of the antigen dilution series response curve and a corresponding concentration is deduced by matching the analyte response to the equivalent intensity in the calibration curve calculated from the calibration array. Thus, an accurate, quantitative and statistically significant result is provided with high confidence.

The graph shown in FIG. 10 represents the results of fixed array processing. Each of the dilution series is plotted (from highest to lowest) and the average of the three series is calculated. Each of the nine capture locations is then plotted against the dilution curves and the average concentration is derived. In addition, data such as min/max, standard deviation and coefficient of variability (+/−CV) can also be reported.

The test dots include reagents that specifically bind to the analyte for which the assay is directed. The reagent is preferably a bound antibody specific for the analyte. The bound antibodies are preferably spaced apart to make each bound antibody available for binding to the test antigen free of stearic hindrance from adjacent antigen complexes.

The results of the assay device FIG. 7, is read and calculated by the reader system of the present invention. To determine the concentration of analyte in a sample, the concentrations of two characteristic assay reagents are predetermined. A relationship between a fluorescent intensity of the fixed test dots in a series of samples with known antigen concentrations is determined. An example of a relationship between fluorescent intensity of test dots and known antigen concentration is a sample is shown in the form of a graph as shown in FIG. 10. Next, a relationship between fluorescent intensity of the calibration dots and the amount of antigen in the calibration dots, determined by using excess detection antibody, as shown in FIG. 10. From FIG. 10, an association between the antigen in the sample and the antigen dot concentration is determined. The calibration curve serves as an array-specific standard curve for the determination of the antigen concentration in the samples. The calibration curve is calculated by the reader system of the present invention based on the light intensities of the calibration dots containing known amounts of analyte.

In the instance of a sample of unknown antigen concentration, the sample is premixed with an excess of detecting antibody. This solution is applied to an assay device such as the assay device shown in FIG. 7. The fluorescent intensity of the test dots is normalized against the calibration curve for that particular analyte to provide a normalized test dot value. This normalized test dot value is then read off the calibration curve shown in FIG. 10 for that analyte to give the concentration of analyte in the sample.

This preferred embodiment applies directly to a format described as detection of “fixed array macro matrices”. In this instance, the analyte/protein complexes are generally much larger than micro-organisms and attached to a substrate. The dots are printed for optimal diameter and as droplets ranging in volume from pico to nano liters. The virtual window format is again of great advantage in that both signal is conserved and x/y positioning of dot matrices is maintained. The reader tracks array position and composition and therefore locates and identifies each dot in any fixed array. Because each dot has a known location and identification, the reader in concert with the assay device, needs only a single label excitation source to generate a detection signal. Fixed Array images are automatically tracked as the initiation point of the array is also the registration of origin.

Another preferred embodiment of the present invention is the use of multiplex array formats. Two predominant formats are used. The first consists of a single test, which is then printed several times, on the same substrate, including both test arrays and calibration arrays. The ability to run these tests simultaneously using a common patient sample, dramatically increases the confidence limit that the test results are in fact correct. Receiver-Operator curves (ROC curves) can reach better than a 99% assurance that the test results are correct. The second format uses multiple arrays of different assays printed on the common substrate, each with multiple calibration arrays. The reader device of the present invention automatically locates, reads and analyzes these arrays with femtomole sensitivity. Because the arrays are located according to x-y co-ordinates, only a single illumination source is required.

### EXAMPLE 12

### Fixed Array to Test for the Presence and Concentration of Specific Proteins

Each unique fixed-array proteomic assay is comprised of two components—a specific Assay Device and a corresponding Analyte Labeling applicator. A calibrated sample amount of the sample is labelled, shaken for 10 seconds and incubated for five minutes in a glass vial. Contained within the labeling chemistry are two main constituents. These are:
Protein-specific antibodies conjugated with a specific-wavelength dye;
An additional dye that provides the operator later with visual confirmation that the sample reading area of the Assay Device is correctly flooded with the test sample.

The proteins of interest are tagged with the conjugated antibodies during the five-minute incubation period.

Once the incubation period is finished, the test operator discards the first two drops and the third is then dispensed onto the sample loading area. The test sample is drawn into the sample viewing area and in so doing is passed through the separation matrix. The separation matrix filters out any sample impurities e.g. blood cells and delivers the test sample onto the test viewing area containing:
Proteins tagged by protein-specific antibodies conjugated with fluorescent dye,
Sample fluid dyed blue for confirmation that the sample viewing area was correctly filled, and
Protein-specific antibodies conjugated with fluorescent dye

The laminar flow of the fluid transfer causes the test fluid to be drawn past and exposed to two sets of protein arrays that are printed on the surface of the array. These are:
Calibration spots, with varied concentrations of the protein of interest, and
Test spots, which contain the capture antibody.

The non-analyte complexed fluorescing antibodies bind to the calibration dots which are printed as a concentration gradient format ranging in concentration of 12.5 μ/ml to 200 μ/ml of human IgG, shown in Illustration 12A. This test array contains 5 calibration concentrations, repeated three times in three separate arrays to provide the basis for automatic calibration of the test. The tagged proteins in the sample fluid are captured by analyte protein-specific antibodies in the test locations as shown in Illustration 12B for a patient's plasma sample being tested for a reaction to Toxoplasma.

Patient Serum Analysis: Fixed Arrays printed in picoliter format. Images were developed by incubating the chip with patient serum, washing, and then incubating with Goat anti-human IgG conjugated to DY47 fluorescent dye. The Assay device was then inserted into the reader of the present invention for automated analysis.

A further embodiment of sequential virtual window array scanning allows the reading of signal from tissue sections. Appropriately labelled tissue samples are investigated, imaged and digitally recorded. All images undergo digital image processing and are optionally stored for record keeping and regulatory purposes.

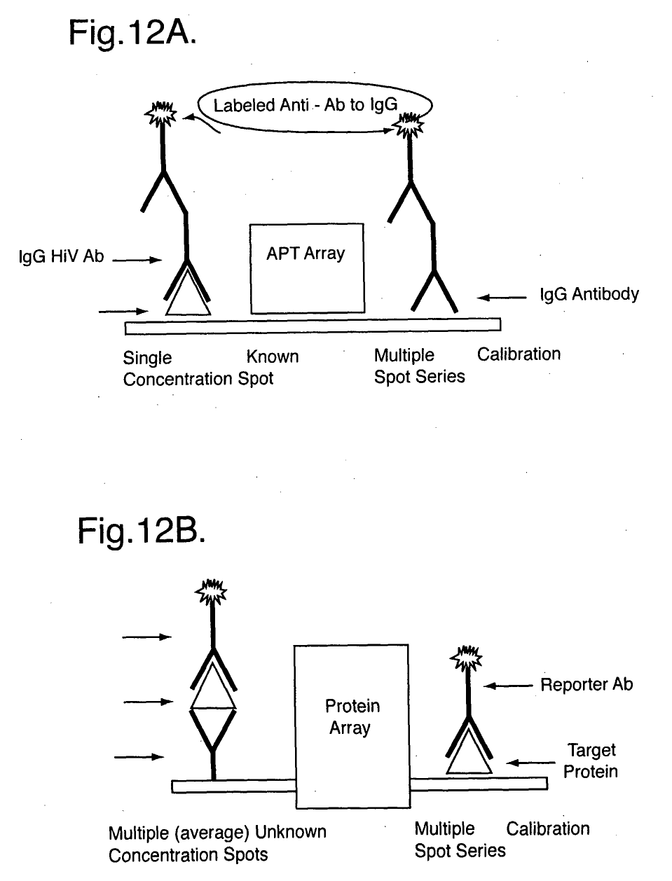

A typical Acquired Pathogen Titer Array (APT), which presents single concentration spots, is made up as in the example shown in FIG. 12A. In this array the specific HIV antigen on the sandwich assay bottom is the target. The middle is a Human IgG HIV antibody specific to the HIV antigen, and the_top reporter labeled with a specific anti antibody to Human IgG. This is in contrast to a standard protein array shown in FIG. 12B, which presents multiple calibration spots, in which the_bottom is generic Human IgG antigen and the_reporter is labeled with a specific anti-Human antibody to Human IgG.

Both types of arrays are read by the reader within the same assay device for each test to obtain accurate quantitative measurement of analyte represented in the molecular aggregates.

### EXAMPLE 13

### Tissue Sections Analysis

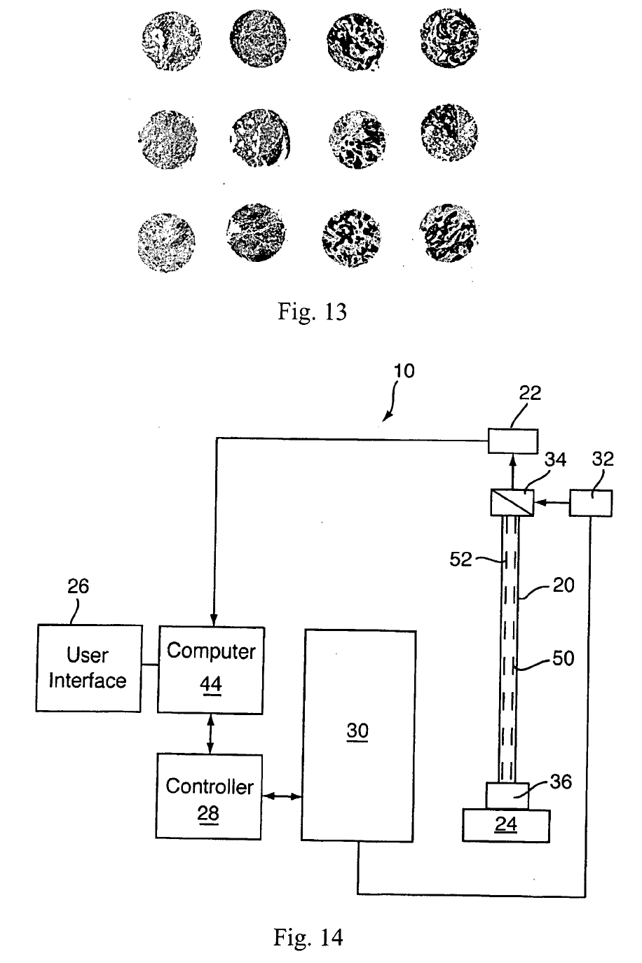

Tissue cores, about 0.5 to 1.5 mm in diameter are punched out of fixed tissue samples and embedded into paraffin blocks. Three cores from each tissue are assembled into an array in a second paraffin block. Sections are cut with a microtome to be arranged in comparative tissue section arrays on an Assay Device. The tissue arrays are attached to the viewing area of the Assay Device of the present invention to be immuno stained for specific markers as shown in FIG. 13.

### EXAMPLE 14

In order for the method of the present invention to have optimal reciprocity with both reader and assay devices the following control parameters constitute an integral sequence for routine auto-analysis.

Printed on the Assay device is an assay-specific identifier. The Reader of the present invention seeks to the specific location of the Assay device containing the assay-specific identifiers and loads any test-specific routines.

The Reader then locates and confirms that the calibration dots have been tagged with the respective antibodies. If an incorrect Analyte Labeler has been used to dispense the sample, the reader will recognize that the test has been compromised and the test will conclude with an appropriate notification message.

Once the positive-control test has completed, the reader proceeds to each test dot and compares the light level of the fluorescing proteins with the level emitted by the calibration dots. Given that the calibration dots are increasing over a dynamic concentration range, the signal to noise ration derived as a function of protein concentration to fluorescence emission intensity, making it possible to determine, with accuracy, the concentration of proteins present in the test sample.

When the sample has been auto-processed, the reader of the present invention performs additional housekeeping tasks. These include:
Making the Assay device un-readable to prevent further use;
The Results are recorded in a log file with:
   The operators ID
   Date and Time
   Test performed
   Test Results

The results are ready for reporting to QA systems or for hard copy printout.

Those skilled in the art will recognize, or be able to ascertain using no more than routine experimentation, many equivalents to the embodiments of the invention described specifically above. Such equivalents are intended to be encompassed in the scope of the following claims.

1. An analyte reading system consisting of a unit for reading and measuring the qualitative and quantitative outcome of an assay in an assay device for a labelled analyte, comprising
an X-Y-Z positioning stage for holding the assay device in a desired location,
a light sensor,
and an optical system comprising
an excitation light source for illuminating a labelled analyte, and
a dichroic mirror for reflecting excitation light to the analyte and emittor radiation to pass through to the light sensor.

2. An analyte reading system according to claim 1, wherein the reader unit further comprises a computer operatively connected to the radiation sensor for receiving a signal from the radiation sensor and performing calculations based on said signal.

3. An analyte reading system according to claim 1, wherein the excitation radiation source is a laser.

4. An analyte reading system according to claim 1, wherein the light sensor is an imaging device.

5. An analyte reading system according to claim 1, further comprising a side illumination means for focussing the optical system on the assay device.

6. An analyte reading system according to claim 1, further comprising a stage controller board for controlling relative location of the positioning stage in three dimensions relative to the optical system.

7. An analyte reading system according to claim 1, further comprising a user interface for communicating to the user the signal detected by the signal recording means and for input by the user of control commands.

8. An analyte reading system according to claim 1, further comprising an automatic initialization and calibration sequence in reference to a calibrated emission standard.

9. An analyte reading system according to claim 1, wherein the reader partitions the assay device reading area into virtual areas of viewing for imaging.

10. An analyte reading system according to claim 9, wherein the reader selects sequential, dedicated viewing areas to examined.

11. An analyte reading system according to claim 10, wherein only the viewing area under examination is irradiated.

12. An analyte reading system for measuring the outcome of an assay in an assay device containing a fluorescently labelled analyte, comprising
a positioning stage for holding the assay device in a desired position,
a light sensor,
an optical system comprising
an excitation light source for illuminating a fluorescently labelled analyte, and
a dichroic mirror for reflecting excitation light to the analyte and light emitted by the fluorescent dye to pass through to the light sensor, and
a computer for processing the signal detected by the light sensor to generate a measurement of analyte concentration on a detected portion of the assay slide.

13. An analyte reading system according to claim 12 wherein the excitation light source supplies full spectrum radiation.

14. An analyte reading system according to claim 13 wherein an excitation wavelength is selected from the full spectrum source radiation.

15. An analyte reading system according to claim 13 wherein the excitation light source is a laser.

16. An analyte reading system according to claim 13 wherein the excitation light source is an ultraviolet light source.

17. An analyte reading system according to claim 12, wherein the light sensor is an imaging device.

18. An analyte reading system according to claim 12, further comprising a side illumination means for focussing the optical system on the assay device.

19. An analyte reading system according to claim 12, further comprising a stage controller board for controlling relative location of the positioning stage in three dimensions relative to the optical system.

20. An analyte reading system according to claim 12, further comprising a user interface for communicating to the user the signal detected by the signal recording means and for input by the user of control commands.

21. A method of reading an assay device containing a fluorescently labelled analyte, comprising the steps of:
a. illuminating a portion of the assay slide containing a test sample.
b. detecting an intensity of light emitted by the test sample in a single image field, and
c. generating a measurement of analyte density in the test sample based on said intensity detection.

22. A method of reading an assay device containing a fluorescently labelled analyte, comprising the steps of:
a. illuminating a portion of the assay slide containing a test sample of unknown analyte density and a portion of the assay slide containing a calibration sample of known analyte density with an excitation light,
b. detecting an intensity of light emitted by the test sample and an intensity of light emitted by the calibration sample in a single image field, and
c. comparing the intensity of light emitted by the test sample to the intensity of light emitted by the calibration sample to generate a measurement of analyte density in the test sample.

23. An analyte reading system according to claim 1, wherein the optical imaging assembly sequentially examines cylindrical fluid volumes of sample located in the viewing area of the assay device.

24. An analyte reading system according to claim 23, wherein the assay device displays three-dimensional random array formats contained in each optical volume.

25. An analyte reading system according to claim 24, wherein the reader counts the particles contained in the random arrays of each optical volume.

26. An analyte reading system according to claim 25, wherein the reader counts the objects of interest contained in the random array matrices of each optical volume.

27. An analyte reading system according to claim 26, wherein the reader sums the random array matrices of all optical volume.

28. An analyte reading system according to claim 27, wherein the reader calculates concentration-per-volume test data.

29. An analyte reading system according to claim 22, wherein the reader measures the fluorescent intensity of fixed array dots as displayed in the viewing area of the assay device.

30. An analyte reading system according to claim 29, wherein the reader measures the fluorescent intensity of fixed array calibration dots as displayed in the viewing area of the assay device.

31. An analyte reading system according to claim 29, wherein the reader measures the fluorescent intensity of unknown fixed macro matrices as displayed in the viewing area of the assay device.

32. An analyte reading system according to claim 29, wherein the microprocessor calculates the analyte concentration in the test sample.

33. An analyte reading system according to claim 29, wherein the reader automatically locates the reference dots of a fixed macro array in the assay device.

34. An analyte reading system according to claim 33, wherein the reader automatically references the location of the remaining fixed macro array dots in the viewing area of the assay device.

35. An analyte reading system according to claim 34, wherein the reader automatically compares dot morphology to a reference dot morphology.

38. An analyte reading system according to claim 35, wherein the reader automatically excludes dots of non-compliant dot morphology.

39. An analyte reading system according to claim 1, wherein the reader automatically locates the fixed, tissue section macro arrays in the assay device.

40. An analyte reading system according to claim 1, wherein the reader is a fully accessible internet device.

41. An analyte reading system according to claim 1, wherein the reader and the assay device have optimal mutually reciprocity.

42. An analyte reading system according to claim 41, wherein the reader automatically confirms the array device identity.

43. An analyte reading system according to claim 42, wherein the reader automatically loads the correct sub-routines to analyze a sample presented in the assay device.
